(19) United States
(12) Patent Application Publication
(10) Pub. No.: US 2021/0252169 A1
(43) Pub. Date: Aug. 19, 2021

(54) Multi-Functional Infrared-Emitting Composites

(71) Applicant: Rutgers, The State University of New Jersey, New Brunswick, NJ (US)

(72) Inventors: Dominik J. Naczynski, Wallington, NJ (US); Mei-Chee Tan, Singapore (SG); Richard E. Riman, Piscataway, NJ (US); Charles Roth, Princeton, NJ (US); Prabhas V. Moghe, Basking Ridge, NJ (US)

(73) Assignee: Rutgers, The State University of New Jersey, New Brunswick, NJ (US)

(21) Appl. No.: 17/234,669

(22) Filed: Apr. 19, 2021

Related U.S. Application Data
(63) Continuation-in-part of application No. 17/079,733, filed on Oct. 26, 2020.
Application No. 17/079,733 is a continuation of application No. 16/378,792, filed on Apr. 9, 2019, now Pat. No. 10,814,017.
Application No. 16/378,792 is a continuation of application No. 14/115,752, filed on Mar. 10, 2014, now Pat. No. 10,286,088.
(60) Provisional application No. 61/483,128, filed on May 6, 2011.
(60) Provisional application No. 61/482,668, filed on May 5, 2011.

(51) Int. Cl. A61K 49/00; A61B 5/00

(52) U.S. Cl. CPC A61K 49/0065; A61B 5/0075; A61B 5/055; A61K 49/0019; A61K 49/0093

(57) ABSTRACT

Compositions for biomedical applications are disclosed, containing infrared-emitting particles, which contain rare earth-elements that emit in the short-wavelength infrared (SWIR) spectrum, where the particles are encapsulated with a biocompatible matrix to form down-converting encapsulated particles and can optionally further include a contrast agent or radiolabel and be used as multimodal imaging agents, where the multimodal-imaging scheme can be selected from optical/MRI, optical/X-ray imaging, optical/CT, optical/PET and combinations thereof. Multimodal imaging methods are also disclosed.

### CROSS-REFERENCE TO RELATED APPLICATIONS

The present application is a Continuation-in-Part of U.S. application Ser. No. 17/079,733, filed on Oct. 26, 2020, which is a Continuation of U.S. application Ser. No. 16/378,792, filed on Apr. 9, 2019, now U.S. Pat. No. 10,814,017, which is a Continuation of U.S. Non-Provisional application Ser. No. 14/115,752, filed on Mar. 10, 2014, now U.S. Pat. No. 10,286,088, which is a U.S. National Phase of International Patent Application Serial No. PCT/US12/36852, filed May 7, 2012, which claims the benefit of priority under 35 U.S.C. § 119(e) of U.S. Provisional Application Ser. No. 61/483,128, filed on May 6, 2011, and 61/482,668, filed on May 5, 2011, the disclosures of which are all incorporated herein by reference.

The present application is also related to U.S. Ser. No. 13/466,079, filed on May 7, 2012, the entire disclosure of which is also incorporated by reference.

### STATEMENT REGARDING FEDERALLY SPONSORED RESEARCH OR DEVELOPMENT

The invention was made with government support under Contract No. NIRT 0609000, awarded by the National Science Foundation. The government has certain rights to this invention.

### BACKGROUND OF THE INVENTION

It is increasingly recognized that molecular and cellular imaging approaches are needed to provide a personalized portrait of disease states and to track responses to therapeutic regimens. Currently, the use of visible-light excitable fluorophores is limited due to poor tissue penetration and autofluorescence caused by the excitation light.

### BRIEF SUMMARY OF THE INVENTION

The present invention is directed to the downconversion properties of the specifically coated rare earth materials. The current state of the art has been focused exclusively on the upconversion properties of rare earths. Applicants now discovered that certain coated rare earth materials can be specifically tuned to optimize shortwave infrared emissions for the purpose of non-invasive infrared imaging applications. Biomedical infrared imaging (i.e., where both excitation and emission is in the infrared) has not heretofore been utilized by either the rare earth or biomedical imaging communities.

It has now been discovered that fluorescence generated by the excitation of Rare Earth elements, hereinafter REs, such as Yb—Er co-doped NaYF₄ nanoparticles, with near infrared light avoids these shortcomings, providing highly useful luminescence for biological imaging. We disclose a novel approach for fabricating water dispersible and biologically targeted REs by encapsulation of the particles in human serum albumin nanoshells ((RE)ANSs)). The encapsulation reduced the cytotoxicity of the REs while providing surface groups for conjugating targeting ligands. (RE)ANSs modified with cyclic-RGD (cRGD) targeted αVβ3 integrin receptors overexpressed on U87 glioblastoma cells with minimal targeting of low αVβ3-expressing cells. Furthermore, upon intravenous injection into mice exhibiting melanoma, the emission of the (RE)ANSs in the 1.5-1.6 μm spectral range can be visualized in lesions throughout the animal using an InGaAs camera. Additional modification of the carriers by therapeutic encapsulation creates multifunctional nanoparticles for both imaging and drug delivery applications. Our results indicate that (RE)ANSs are suitable for imaging cancer cells, as we all for combined imaging and targeted drug delivery in vivo. Additional shell compositions can include one or more of polypeptides, polysaccharides such as glucose and dextran, biocompatible polymers, and exosomes, as well as albumin.

One embodiment of the invention is directed to methods of preparation of rare earth-based light-emitting multifunctional composites, and their use in biomedical applications, such as non-invasive imaging (2-dimensional and 3-dimensional), image-guided interventions (surgical and non-surgical), drug tracking and delivery and photodynamic therapy. In contrast to current optical imaging contrast agents, these infrared-emitting rare earth composites are activated by near infrared sources (700 to 1000 nm) to emit also in the short-wave infrared (e.g. 1000 to 2500 nm) and visible wavelength radiation ranges (400 to 700 nm).

The infrared-emitting composites contain of one or more infrared-emitting rare earth doped nanoparticles ((RE)NPs) encapsulated within a biocompatible polymer, polypeptide, polysaccharide or macromolecule (e.g., deoxyribonucleic acid, ribonucleic acid, proteins, glycoproteins). The size range of these composites can be tailored from 20 nm to 10 μm, and can be modified using different polymers, polysaccharides, polypeptides, macromolecules, or exosomes to control the in vivo bio-distribution. Various embodiments of the invention serve multifunctional utility in a variety of therapeutic management strategies. For therapeutic applications, the composite can also serve as a drug carrier (drug refers to any pharmacologic factor such as a small bioactive molecule or gene or biologic), where the biophotonic properties of the carrier can be used to track the drug distribution and release or to actuate drug release by light emitted from the rare earth nanoparticles or controlled by the polymer, polysaccharide, polypeptide or macromolecule degradation. Further functionality to the composite can be added by modifying the surfaces with targeting ligands to localize these carriers to specific sites of interest.

One embodiment of the present invention is directed to a method of non-invasive infrared imaging, including the steps of:
(a) administering a composition containing infrared-emitting nanoparticles containing rare earth elements, wherein the nanoparticles are encapsulated with a biocompatible matrix to form down-converting coated nanoparticles tuned to optimize short-wavelength infrared (SWIR) emission; and
(b) irradiating with infrared radiation, wherein both excitation and emission spectra of the coated nanoparticles are in the infrared region.

Another embodiment of the invention is directed to a method of image-guided biomedical intervention, including the steps of:
(a) administering a composition containing infrared-emitting nanoparticles containing rare earth elements, wherein the nanoparticles are encapsulated with a biocompatible matrix to form down-converting coated nanoparticles tuned to optimize short-wavelength infrared (SWIR) emission; and
(b) irradiating with infrared radiation, wherein both excitation and emission spectra of the coated nanoparticles are in the infrared region.

Still another embodiment of the invention is directed to a method of drug tracking and delivery, including the steps of:
(a) administering a drug composition containing infrared-emitting nanoparticles containing rare earth elements, wherein the nanoparticles are encapsulated with a biocompatible matrix to form down-converting coated nanoparticles tuned to optimize short-wavelength infrared (SWIR) emission; and
(b) irradiating with infrared radiation, wherein both excitation and emission spectra of the coated nanoparticles are in the infrared region.

Another embodiment of the invention is directed to a method of improving the biocompatibility and/or reducing the toxicity and/or side effects of a drug and/or imaging agent, including the step of encapsulating the drug and/or imaging agent with a biocompatible matrix.

Yet another embodiment of the invention is directed to a method of biologically targeting a drug and/or imaging agent, including the step of encapsulating the drug and/or imaging agent with a biocompatible matrix.

For any of the above methods, a preferred biocompatible matrix includes human serum albumin (HSA). One or more of polypeptides, polysaccharides such as glucose and dextran, biocompatible polymers, and exosomes can be used instead of or in addition to HSA. In addition, for any of the above methods, the biocompatible matrix can further include a pharmaceutical agent, and/or a targeting molecule, which directs the coated nanoparticle to a biological target. A preferred targeting molecule includes cyclic arginine-glycine-aspartic acid (cRGD) tripeptide.

According to one embodiment, SWIR-emitting particles according to the present invention are provided as a host material doped with one or more SWIR-emitting rare earth elements. Compositions according to the present invention include host materials doped with one or more rare earth elements selected from Yb, Nd, Pm, Sm, Eu, Gd, Tb, Tm, Er, Pr, Dy and Ho. According to an embodiment, the rare earth elements are selected from Pr, Nd, Yb and Er.

Host materials suitable for use in the present invention must be transparent to both excitation and emission wavelengths. Short-wave infrared (SWIR) light is typically defined as light in the 0.9-1.7 μm wavelength range, but can also be classified from 0.7-2.5 μm. Host materials according to the present invention at least should be transparent at about 1.5 mm. This can include both low and high phonon energy host materials, although low phonon energy materials will minimize non-radiative energy loss. Suitable high phonon energy host materials include oxyhalides, aluminates, mixed halide-oxides such as LaOCl, fluoroxide glasses, oxides such as borates, germinates, silicates, carbonates, phosphates, and the like, other chalcogenides, including metal chalcogenides such as metal sulfides and metal tellurides, other oxygen-containing phosphorus compounds, such as phosphonates and pentaphosphates, and the like. Suitable low phonon energy host materials include low phonon energy halides.

The dopant and host material should be matched to provide a combination where the host material, whether it is a high or low phonon energy material in transparent to the emission wavelength or wavelengths of the dopant. Combination selections can be readily performed by one of ordinary skill in the art without undue experimentation. According to an embodiment, compositions are provided based on halide hosts selected from BaF₂, NaYF₄, YF₃, LaF₃, CeF₃, CaF₂ and CsCdBr₃.

According to another aspect of the present invention, compositions are provided that can be used in multi-modal imaging methods. According to one embodiment, multi-modal imaging compositions are provided containing a plurality of infrared-emitting particles that contain rare earth-elements that emit in the short-wavelength infrared (SWIR) spectrum, wherein the particles optionally further include a radiolabel for PET imaging, or one or more contrast elements for MRI, CT or X-ray radiographic imaging, or both a radiolabel and a contrast element, wherein the infrared-emitting particles are directly encapsulated with a shell characterized by one or more encapsulants selected from polypeptides, polysaccharides, biocompatible polymers, and exosomes, to form spherical down-converting microcapsules containing a plurality of the infrared-emitting particles, wherein the infrared-emitting particles have a size between 2 nm and 10 micrometers, the microcapsules have a capsule size between 10 nm and 100 micrometers, and the infrared-emitting particles have a relative size permitting the plurality of infrared-emitting particles to be loaded into the microcapsules.

Nanoparticles can further include one or more Rare Earth elements (REs) contrast agents selected from Er, Dy, La, Ce, Pm, Sm, Eu, Gd, Tb, Yb, Nd, Tm, Pr, Ho and Lu. These elements can serve a dual role in a multi-modal imaging nanoparticle composition and can be used alone or optionally in combination with other contrast agents and other SWIR-emitting rare earth elements.

Multimodal imaging methods and compositions according to the present invention include multimodal imaging methods wherein the compositions of the preset invention include a contrast agent for Magnetic Resonance Imaging (MRI), Computerized Tomography (CT) scanning and X-ray radiography. Positive Emission Tomography (PET) radiolabels can be included as well.

Therefore, according to another embodiment of the present invention, a method of non-invasive multimodal imaging of a subject is provided that includes the steps of:
a) administering to a subject a multi-modal imaging composition according to the present invention, and
b) subjecting the subject to a multimodal imaging scheme selected from optical imaging in combination with an imaging method selected from MRI, CT, X-ray radiography and combinations thereof, wherein the composition includes a contrast agent, and/or a PET imaging method, wherein said composition includes a radiolabel.

Any non-toxic compound with sufficient electrons can be suitable for use as a contrast agent. The elements of Period 5 on the periodic table and the rows below, i.e., Periods 5 to 7, can be used as long as they are not toxic. For example, Ce, Y, Sr, meet both conditions. Many others meet these conditions as well, so that the entire Period 6, including REs (Lanthanides), and the entire Period 7, including Actinides all have sufficient electrons within the atom.

In one embodiment, host materials and/or RE dopants are selected to provide a multimodal composition that serves as both a contrast agent and a SWIR-emitting nanoparticle. In one embodiment, nanoparticles can further include one or more contrast elements selected from Er, Dy, La, Ce, Pm, Sm, Eu, Gd, Tb, Yb, Nd, Tm, Pr, Ho and Lu. When Er and Dy are used, these elements serve a dual role in a multi-modal imaging nanoparticle composition and can be used alone or optionally in combination with other contrast imaging elements. In another embodiment, contrast agent host materials are chosen that are doped with SWIR-emitting REs to provide dual role contrast agent/SWIR-emitting nanoparticles.

The present invention may also include Positive Emission Tomography (PET) radiolabels. When additional contrast elements and radiolabels are used, they can be part of the SWIR-emitting nanoparticle composition or part of the shell composition (e.g., glucose, albumin, dextran, polypeptides, etc.). Embodiments according to the present invention include embodiments wherein the radiolabeling of the SWIR-emitting particles for PET contrast are part of the composition host material (e.g., F¹⁸) present within the SWIR-emitting particles (i.e., not an additional layer) and embodiments where the radiolabel (e.g., O¹⁵) can be included as part of the shell composition (e.g., glucose, albumin, dextran, polypeptide, etc.). When the host material is radiolabeled, the multimodal nanoparticles of the present invention simultaneously serve three roles as a radiolabel, contrast agent and SWIR-emitting nanoparticle.

The present invention thus provides multimodal imaging methods selected from optical/MRI, optical/X-ray radiography, optical/CT, optical/PET and combinations thereof.

Yet another embodiment of the invention is directed to a multi-modal imaging composition for biomedical applications, including infrared-emitting nanoparticles including rare earth-elements, wherein the nanoparticles are encapsulated with a biocompatible matrix to form downconverting coated nanoparticles tuned to optimize short-wavelength infrared (SWIR) emission. Preferably the biocompatible matrix includes human serum albumin (HSA). Also, the biocompatible matrix can further include a pharmaceutical agent, and/or a targeting molecule which directs the coated nanoparticle to a biological target. A preferred targeting molecule includes cyclic arginine-glycine-aspartic acid (cRGD) tripeptide.

As above, the nanoparticles include the above-disclosed low phonon energy halide host materials, such as CeF₃, doped with one or more rare earth elements selected from Yb, Nd, Pm, Sm, Eu, Gd, Tb, Tm, Er, Pr, Dy and Ho. Preferably, the rare earth elements are selected from Pr, Nd, Yb and Er. In addition, the nanoparticles can further include one or more of the above-described contrast agents, such as elements selected from Er, Dy, La, Ce, Pm, Sm, Eu, Gd, Tb, and Lu. These elements can be part of the SWIR-emitting composition or part of the shell composition (e.g., glucose, albumin, dextran, polypeptides, etc.). Er, Nd, Tm, Pr, Ho and Dy, optionally combined with Yb, can be serve dual roles in optical/MRI, optical/CT and X-ray radiography.

The multimodal imaging scheme can be selected from optical/MRI, optical/X-ray radiography, optical/CT, optical/PET and combinations thereof. The radiolabeling of SWIR emitting particles for PET contrast can be part of the composition (e.g., F¹⁸) present within the SWIR-emitting particles (i.e., not an additional layer). The radiolabel (e.g., O¹⁵) can also be included as part of the shell composition (e.g., glucose, albumin, dextran, polypeptides, etc.)

### BRIEF DESCRIPTION OF THE DRAWINGS

FIGS. 1A, 1B, 1C and 1D show physical (FIGS. 1A and 1B) and optical (FIGS. 1C and 1D) properties of REs.

FIG. 2 displays a schematic representation of the method of albumin encapsulation of rare earth nanoparticles.

FIG. 3 illustrates a demonstration of human glioblastoma cells targeted without (top) & with (bottom) cyclic RGD presenting nanoparticles.

FIG. 4 illustrates a SWIR imaging set-up of an anesthetized mouse displaying pigmented melanoma following (RE)ANS injection.

FIG. 5 displays the SWIR emission spectra of variously sized (RE)ANS following 980 nm excitation.

FIGS. 6A and 6B show a picture of a mouse exhibiting melanoma lesions (FIG. 6A) injected with 100 μl of (RE)ANS (2.0 mg RE ml⁻¹) via the peritoneum and imaged for SWIR emission after 30 minutes (FIG. 6B).

FIGS. 7A, 7B, 7C, 7D, 7E, 7F, 7G and 7H show pictures of mice exhibiting melanoma lesions (FIGS. 7A and 7E) injected with 100 μl of REs (FIGS. 7B, 7C and 7D) and (RE)ANS (2.0 mg RE ml⁻¹) (FIGS. 7F, 7G and 7H) via the peritoneum.

FIG. 8 shows a picture of a mouse exhibiting melanoma dissected after 168 h following IP injection of REs (100 μl at 2.0 mg RE ml⁻¹).

FIGS. 9A, 9B and 9C show drug loading of variously sized ANS with curcumin (FIG. 9A and FIG. 9B) and binding efficiency of loading (FIG. 9C).

FIGS. 10A, 10B and 10C show drug loading of ANS with BAY 36-7620 (FIG. 10A) and cytotoxicity assay of LZ-83 murine (FIG. 10B) and WM239A human (FIG. 10C) melanoma cells exposed to ANSs with and without BAY 36-7620, a glutamate receptor antagonist shown to induce cell death in numerous melanoma cell lines.

FIGS. 11A, 11B and 11C display physical (FIGS. 11A and 11B) and optical (FIG. 11C) properties of (RE)ANCs.

FIGS. 12A and 12B show the transmission efficiency (FIG. 12A) and absorbance (FIG. 12B) of SWIR and visible RE emissions through blood and pigmented tumor samples.

### DETAILED DESCRIPTION OF PREFERRED EMBODIMENTS

Rare Earth (RE) Nanoparticles as a Superior Modality as an IR-Emission Platform

Using REs for 2- and 3-dimensional non-invasive imaging presents several unique, competitive advantages over conventional imaging agents. The wavelengths of emissions can be tailored by controlling the nature and concentration of the dopant and host. Infrared excitation enables deeper tissue penetration depths and low background tissue autofluorescence. Also, since there is no overlap between the excitation and emission wavelengths, images with high signal-to-noise ratio can be collected using RE nanoparticles.

The extent of infrared light propagation and the volumetric energy distribution of infrared light are governed by the absorption and scattering properties of tissues. Major tissue absorbers of infrared light are hemoglobin, melanin and water, while composition, size and morphology of tissue components control the light scattering. Infrared light has been reported to travel through 10 cm of breast tissue and 4 cm of skull tissue using microwatt sources. Based on absorption properties, the attenuation coefficients of oxygenated blood in visible and infrared regions are 500 and 30 cm⁻¹, respectively, which corresponds to attenuation length of 0.002 and 0.03 cm where the intensity of the beam has dropped to 1/e. Therefore, infrared light can reach penetration depth of up to almost 10 times that of visible light.

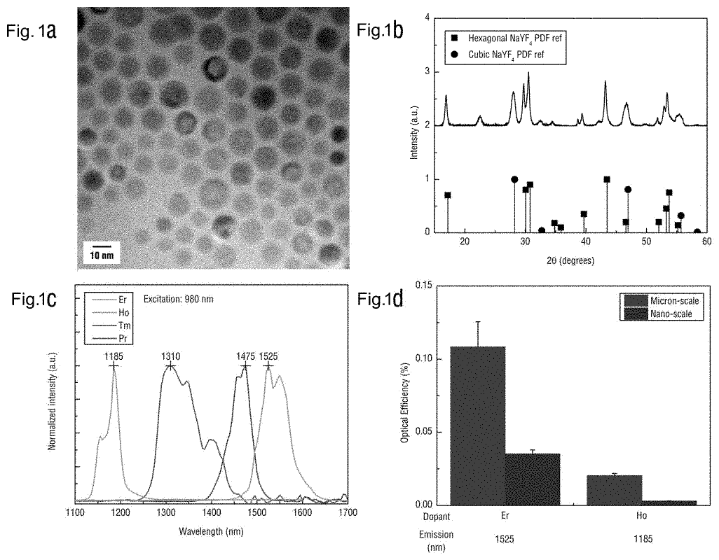

Most REs can be tuned to emit in both the visible and infrared regions (see FIGS. 1A-1D). Using infrared emissions for imaging have been reported to improve imaging sensitivity by up to ˜10 times. In addition, REs are more stable than organic dyes, showing almost no loss in emission intensities over time (e.g. 30-60 days). In comparison, most organic dyes suffer from poor photostability, which results in reducing emission intensities after a day. Alternative possible infrared-emitting inorganic substitutes like HgTe and CdₓHg₁ₓTe, InP and InAs, and PbS, PbSe, and PbTe include several well-known toxic elements, and are thus not favorable for biomedical applications.

Weakly infrared emitting carbon nanotubes and gold particles have broad emission peaks (i.e. bandwidth>>100 nm) and typically require an optimal excitation in the visible region or high power pulsed excitation sources (˜20 W). In contrast, the optimal excitation of rare earth nanoparticles ((RE)NPs) have narrow emission bandwidths (<100 nm) and can be tuned to be excited within the NIR window using low power continuous wave sources (<2 W).

Rare Earth/Biocompatible Matrix Composites

A drug delivery composite platform that utilizes near infrared both “upconversion” and “downconversion” fluorescence, providing highly useful luminescence for biological imaging both in vitro and in vivo has been discovered. The final encapsulated composite material is stable in aqueous solution, highly biocompatible and is amenable to fluorescence imaging with high fidelity.

Typically, naked (i.e., uncoated) REs are not suitable for biological applications due to their insolubility and tendency to agglomerate in aqueous solution. Additionally, naked REs are limited by a lack of functional groups for surface attachment of ligands or other biomolecules for actively targeted delivery, and may potentially have dose- and time-dependent cytotoxic effects. Many of the existing coating methods have issues of toxicity, poor stability in water, or require numerous steps to create functional groups on the particle surface for further conjugation to bio-molecules. A chemical functional group is defined as a submolecular structural motif, characterized by specific elemental composition and connectivity, that confers chemical reactivity upon the molecule that contains it. Examples of useful chemical functional groups include, without limitation: hydroxyl, carboxyl, amine, alkyl and thiol groups.

Biocompatibility is conferred or enhanced by coating the particles with a biocompatible matrix. Enhanced biocompatibility can be manifested in reduced in vitro or in vivo toxicity. In one embodiment of the invention, rare earth-containing particles, preferably nanoparticles (REs) are encapsulated to form rare earth-containing particles coated with an biocompatible matrix that is optically transparent. Optical transparency of the matrix is important to the excitation and emission properties of the composites. Encapsulation can result in the coating of a single particle, or can be adjusted by those skilled in the art to provide encapsulated composites including a plurality of particles encapsulated in a nanoshell. The particle loading in such a particle encapsulate can be 0.004 to 94 weight % (0.001 to 80 volume %). A composite including a multiparticle microcapsule is preferred for biomedical applications due to the phonon-assisted energy transfer between the particles.

The ratio of the biocompatible matrix to rare earth-containing particles can be adjusted based on the desired therapeutic action. The multiparticle microcapsule can include a low concentration of heavily rare earth-doped particles, or a high concentration of lightly rare earth-doped particles. Conversely, the multiparticle microcapsule can include a low concentration of lightly rare earth-doped particles, or a high concentration of heavily rare earth-doped particles. For biomedical applications the highest weight percent load of particles in the matrix is generally about 10 to 40 weight % (2.6 to 13.7 volume %). The biocompatible matrix can be covalently bound to the particles, or non-covalently associated.

Biocompatible matrix compositions include one or more of polypeptides, polysaccharides such as glucose and dextran, and biocompatible polymers, as well as albumin Examples of suitable macromolecules, polypeptides, polysaccharides or polymers that impart similar benefits as seen with albumin include without limitation, poly-L-lysine, poly-D-lysine, poly-ethylene glycol [PEG], poly-2-hydroxyethyl aspartamide, poly(d,l-lactide-co-glycolide) [PLGA], poly(methyl methacrylate) [PMMA], poly(N-isopropylacrylamide), poly(amidoamine) [PAMAM], poly(ethyleneimine), poly lactic acid, polycaprolactone, dextran, alginates, chitosan, transferrin, collagenase, polydopamine, 1,2-distearoyl-sn-glycero-3-phosphoethanol-amine-N-[amino(polyethylene glycol)-2000 [DSPE-PEG], and gelatin. The macromolecules, polypeptides, polysaccharides or polymers are attached to rare earth-containing particles through either physical or chemical bonds (e.g., covalent, van der Waals, ionic, electrostatic, hydrogen bonds).

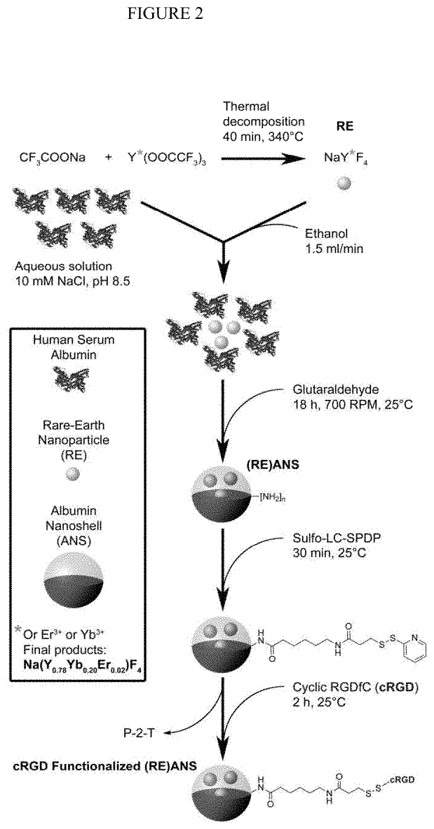

The rare earth-containing particles themselves have a particle size between 2 nm and 100 micrometers (microns), preferably between 5 nm and 10 micrometers, more preferably between 10 nm and 1 micrometer, most preferably between 10 nm and 500 nm. Nanoparticles are preferred. Encapsulation of particles can be achieved using controlled coacervation of the biocompatible matrix, for example, human serum albumin (HSA), in an aqueous solution to provide multiparticle microcapsules having a particle size between 5 nm and 100 micrometers, preferably between 10 nm and 1 micrometer, more preferably between 10 nm and 500 nm, and most preferably between 100 nm and 300 nm (see FIGS. 1A-1D and FIG. 2). One skilled in the art can elect the size of the particle encapsulate/multiparticle microcapsule based on the desired therapeutic action. The particles to be encapsulated are smaller than the multiparticle microcapsule. In some embodiments the particles to be encapsulated are about a factor of 10 or more smaller than the multiparticle microcapsule. Nanoparticle encapsulates are generally preferred.

In order to form the particles of the invention, host particles are doped with at least one rare earth element that emits in the short-wavelength infrared (SWIR) spectrum. The SWIR spectrum covers the wavelength range of about 1.4 to about 3 micrometers (microns). The rare earth doping concentration can be as low as 1 ppb, and as high as the concentration where concentration or quantum quenching begins, which depends on both the host particle and the rare earth element, but is generally about 7.5 mole percent at the high end (for example, Yb in CeF₃). The rate at which concentration quenching begins is readily determined by those skilled in the art. Preferably the doping concentration ranges between 1 ppm and 5 mole percent. More preferably the doping concentration ranges between 1000 ppm and 5 mole percent. Still more preferably the doping concentration ranges between 0.1 and 3 mole percent. Most preferably the doping concentration ranges between 0.5 and 1 mole percent.

Host materials suitable for use in the present invention must be transparent to both excitation and emission wavelengths. Short-wave infrared (SWIR) light is typically defined as light in the 0.9-1.7 μm wavelength range, but can also be classified from 0.7-2.5 μm. Host materials according to the present invention at least should be transparent at about 1.5 mm. This can include both low and high phonon energy host materials, although low phonon energy materials will minimize non-radiative energy loss. Suitable high phonon energy host materials include oxyhalides, aluminates, mixed halide-oxides such as LaOCl, fluoroxide glasses, oxides such as borates, germinates, silicates, carbonates, phosphates, and the like, other chalcogenides, including metal chalcogenides such as metal sulfides and metal tellurides, other oxygen-containing phosphorus compounds, such as phosphonates and pentaphosphates, and the like. According to an embodiment, compositions are provided based on halide hosts selected from BaF₂, NaYF₄, YF₃, LaF₃, CeF₃, CaF₂ and CsCdBr₃. A preferred host is CeF₃. US Application Publication No. 2013/0032759 discloses suitable rare earth-doped particles, and is incorporated herein by reference in its entirety.

The doped particles of the invention are preferably highly chemically uniform in terms of the distribution of the rare earth element(s) in the host. This prevents localized concentration quenching. One method of uniform synthesis is taught in D. J. Naczynski, et al., “Albumin nanoshell encapsulation of near infrared excitable rare-earth nanoparticles enhances biocompatibility and enables targeted cell imaging”, Small, 6 [15] 1631-1640 (2010).

The crystalline phase of the particles is selected which results in the greatest intensity emission. This is readily done by those skilled in the art. FIGS. 1A-1D show the physical and optical properties of REs. The TEM images of REs (a) show uniform 10 nm spherical particles. X-ray crystallography (XRD) plot of REs show a predominately hexagonal phase crystalline structure (b). Hexagonal phase REs exhibit the most efficient and intense SWIR emissions. The SWIR (c) emission of REs can be tuned by changing the types of rare earth dopant used during synthesis. REs consisting of a NaYF₄ host doped with ytterbium (Yb) and one or more elements selected from erbium (Er), holmium (Ho), thulium (Tm) and praseodymium (Pr) are favored for their low phonon energies that minimize non-radiative losses to enable intense emission spanning the SWIR region. The optical efficiency of a specific dopant scheme can be further tuned by varying the particle size of the NaYF₄ host. Generally, larger, micron sized NaYF₄ host exhibits greater SWIR emission than their nanoscale counterparts, with Er and Ho dopant schemes showing the highest optical efficiencies.

One embodiment of the invention involves coating of RE nanoparticles with albumin shells. Human serum albumin nanoshells (ANSs, alternatively termed albumin nanocarriers, ANCs) have been demonstrated to be bio-compatible with many cell types, exhibit long half-life in vivo, are capable of delivering a number of biologically relevant compounds and have numerous functional entities available for conjugating ligands, antibodies and other peptides which can bind to specific molecular receptors. A ligand is a substance that forms a complex with a biomolecule, and binds to a site on a target protein or receptor. The ligand can also serve as a signal triggering molecule that initiates a casacade of biochemical reactions (e.g., cell death). Pharmaceutical formulations composed of human serum albumin are also in use clinically. For example, ABI-007 (ABRAXANE®, Abraxis BioScience Inc, Los Angeles, Calif.) is a commercial, albumin-bound paclitaxel delivery vehicle.

One embodiment of the invention is directed to REs encapsulated within an albumin nanoshell structure. In doing so it is possible to impart to the REs many of the benefits afforded by the ANS system. Once encapsulated, it is possible to functionalize the composite nanoparticle ((RE)ANS) with the cyclic arginine-glycine-aspartic acid (cRGD) tripeptide to examine the tissue targeting and biophotonic properties of the nanocomposites. The composite nanoparticles are observed to be highly biocompatible in vitro, as evidenced by lack of cytotoxicity, and are capable of selectively targeting cancer cell lines that exhibit higher expression of cancer-specific integrin markers, and amenable to fluorescence imaging with high fidelity.

Encapsulation techniques can include coacervation, coprecipitation, solvent evaporation, interfacial polymerization, emulsion, reverse microemulsion, electroporation, thermal shock, saponin-assisted loading, diffusion-based methods (e.g., dialysis, sonication), transfection, and hot melt processes. The method of executing the formulation is crucial to the final composite properties and function. In particular, the electroporation, thermal shock transfection, saponin-assisted loading, and diffusion-based strategies are for encapsulation within exosomes. Transfection can be achieved when the surfaces of the (RE)NPs are modified with either RNA or DNA structures.

Briefly, encapsulation in the form of (RE)ANS attenuated RE-induced cytotoxicity at 2.5 μg ml⁻¹ concentrations and significantly improved the biocompatibility of the rare earth nanoparticles at 250 mg ml⁻¹ after 24 h incubation with various types of human cells. One embodiment of the invention is directed to imaging cells incubated with (RE)ANS for approximately 5 days, after which time it is still possible to observe fluorescence from the cross-linked albumin, indicating that the albumin shell has maintained its integrity over this time course. The long-term cytotoxicity studies of nanoparticle exposure support these observations.

Functionalization of the particles with a targeting ligand was demonstrated with cyclic arginine-glycine-aspartic acid (cRGD) to selectively target cells that over-express the αᵥβ₃ integrin receptor. The αᵥβ₃ is an attractive targeting marker for locating and identifying cancer cells. Antagonists of αᵥβ₃, such as the cRGD tripeptide motif, are capable of blocking both tumor cell metastasis and angiogenesis while providing a means of targeting cancer cells expressing the integrin.

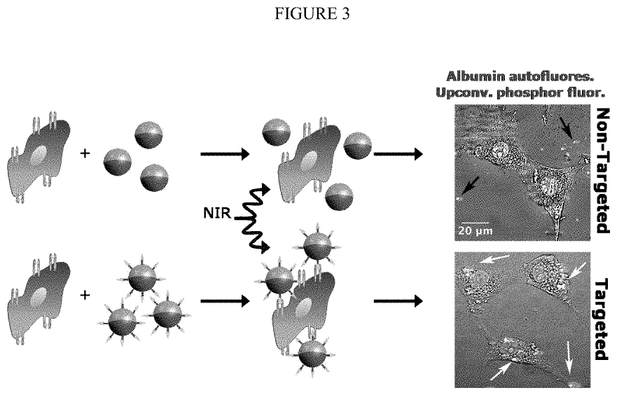

Upon surface modification of (RE)ANS with the cRGD tripeptide as a targeting ligand, the composite particles were demonstrated to be capable of selectively targeting both human glioblastoma and melanoma cell lines exhibiting higher expression of cancer-specific integrin markers, thereby demonstrating the preservation of the molecular potency of the tripeptide motif. (RE)ANS can be functionalized with various cyclic tripeptides using a standard crosslinking procedure conjugating the free amine groups on the albumin shell to thiol groups present on the ligands. (RE)ANS can be functionalized with either cRGD, which targets the integrin αᵥβ₃, or, as a negative control, cyclic arginine-alanine-aspartic acid (cRAD) which does not have affinity for αᵥβ₃. To confirm receptor specific targeting, (RE)ANS functionalized with cRGD were incubated with highly αᵥβ₃ expressing U87-LUC and with low αᵥβ₃ expressing A172 cells. Near infrared-excited fluorescent images of cRGD functionalized particles incubated with the U87 cells show particles distributed in a punctate pattern primarily throughout the cellular cytoplasmic space (FIG. 3). The U87 cells treated with non-functionalized or cRAD functionalized (RE)ANS showed little particle accumulation. Fluorescent images of the A172 cells incubated with cRGD particles demonstrated no detect-able level of association of particles with cells, and appeared similar to the images of A172 cells treated with cRAD functionalized (RE)ANS or un-functionalized (RE)ANS. Quantification of fluorescence within the cell body was significantly elevated in U87-LUC cell line treated with cRGD functionalized (RE)ANS over 4 h in relation to that seen in the A172 cells.

Particles were also incubated with WM239A human melanoma cells, which are known to express high levels of the αᵥβ₃ integrin. After 4 h, the (RE)ANS functionalized with cRGD accumulated around the WM239A cells whereas non-functionalized and cRAD conjugated particles showed low levels of cellular association, as was seen with the U87-LUC cells. Further, cRGD functionalized particles exhibited significantly more cellular internalization compared with non-functionalized particles during the same incubation times, which was confirmed by electron microscopy.

A bio-benign and tissue-targetable composite including nanoscale and submicroscopic scale rare earth doped particles ((RE)NPs) encapsulated in polypeptides (e.g., human serum albumin), polysaccharides, polymers, or exosomes was imbued with superior biophotonic properties, specifically related to emission in the visible (400 to 700 nm) and shortwave infrared (SWIR) window (1000 to 2500 nm) when excited in the near infrared (NIR) window (700 to 1000 nm). “Exosomes” are small bilayer vesicles which are typically produced in the endosomal compartment of most eukaryotic cells, and include both natural exosomes as well as synthetic exosomes. Exosomes are used as a delivery platform due to low toxicity, excellent structural stability, nanoscale size, cargo loading ability, and editable surface structure.

The present invention provides the ability to be optically detected through the NIR excitation of the rare earth-containing particles to generate SWIR emission using low power (<2 W) excitation sources. Both the NIR and SWIR windows have very favorable biomedical optical characteristics, such as the ability to penetrate deeper than visible or UV radiation through biological tissue with minimal scattering and generating low background autofluorescence. NIR and SWIR radiation are also considered to be safe and non-phototoxic, while the composite formulation mitigates the intrinsic cytotoxic properties of RE particles.

The excitation and emission wavelengths of these composites can be tuned by controlling the dopant and host chemistry of rare earth-containing particles, and the density and type of rare earth-containing particles encapsulated by the polypeptide or polymer. The rare earth-containing particles that are incorporated within the composites can be of different sized (e.g. nanoscale or microscale) and morphologies (e.g. spheres, rods, platelets, prisms, cubes, acicular). The absorption and emission properties of rare earth-doped phosphors can be tailored by controlling the local environment, such as site symmetry, crystal field strength and electron-phonon interaction strength of rare earth dopants (see FIGS. 1A-1D). Halide hosts (e.g. BaF₂, NaYF₄, YF₃, LaF₃, CeF₃, CaF₂ and CsCdBr₃) have low phonon energies that minimize non-radiative losses to enable intense SWIR emissions.

For purposes of the present invention, “low phonon energy” is defined as a phonon energy below about 450 cm⁻¹, preferably below about 400 cm⁻¹. Phonon energy ranges from 160 to 1400 cm⁻¹. See K. Soga, et al., “Luminescent properties of nanostructured Dy³⁺- and Tm³⁺-doped lanthanum chloride prepared by reactive atmosphere processing of sol-gel derived lanthanum hydroxide”, J. Appl. Phys., 93 [5] (2003) 2946-2951. CsCdBr₃ is one of the preferred host materials, with a phonon energy of 160 cm⁻¹. The higher phonon energy hosts have less bright emissions, or the emission can be absent altogether. Those skilled in the art are able to predict which combinations of host and rare earth element will be brighter. For example, the 1.3 micrometer emission is absent in oxides, but present in fluoride host materials. However a 1.5 micrometer emission can be found in both oxide and fluoride hosts. For the purposes of the present invention, CeF₃ is a preferred host.

Low phonon energy halide hosts typically exhibit weak bonding and contain atoms of both low and large mass. In a host lattice containing different cations and anions, weak bonding and vastly different masses of the anion and cation are important. For example, diamond, carbon nitride, aluminum nitride and silicon carbide all have very similar atomic masses for the atom pairs, and also have very strong bonds, so that these lattices serve as very poor hosts for SWIR emission; the atoms are also very light, and therefore have poor contrast properties as well. Conversely, materials such as LaCl₃ have weak bonds and very large atomic weight differences between Cl and La. Further, boron oxide is a poor host, but lanthanum borate or yttrium borate can serve as suitable hosts for some SWIR emissions, and also provide good electron contrast. Thus, by definition, low phonon energy halide hosts contain elements that provide high contrast for X-ray, CT and MRI imaging methods. Therefore, host materials such as BaF₂, NaYF₄, YF₃, LaF₃, CeF₃, CaF₂ and CsCdBr₃, and the like, all serve as X-ray, CT and MRI contrast agents. The contrast agent does not have to be a rare earth element.

Multimodal methods and compositions according to the present invention include multimodal imaging methods wherein the compositions of the preset invention include a contrast agent for MRI, CT scanning and X-ray radiography. Positive Emission Tomography (PET) radiolabels can be included as well.

Any non-toxic compound with sufficient electrons can be suitable for use as a contrast agent. The elements of Period 5 on the periodic table and the rows below, i.e., Periods 5, 6 and 7, can be used as long as they are not toxic. For example, Ce, Y, Sr, meet both conditions. Many others meet these conditions as well, such that the entire Period 6, including REs (Lanthanides), and the entire Period 7, including Actinides all have sufficient electrons within the atom.

In one embodiment, host materials and/or RE dopants are selected to provide a multimodal composition that serves as both a contrast agent and an SWIR-emitting nanoparticle. In one embodiment, nanoparticles can further include one or more contrast elements selected from Er, Dy, La, Ce, Pm, Sm, Eu, Gd, Tb, Yb, Nd, Tm, Pr, Ho and Lu. When Er and Dy are used, these elements serve a dual role in a multi-modal imaging nanoparticle composition and can be used alone or in combination with other imaging elements. In another embodiment, low phonon energy contrast agent host materials are chosen that are doped with SWIR-emitting REs to provide dual role contrast agent/SWIR-emitting nanoparticles.

The composition and methods of the present invention may also include Positive Emission Tomography (PET) radiolabels. When additional contrast elements and radiolabels are used, they can be part of the SWIR-emitting nanoparticle composition or part of the shell composition (e.g., glucose, albumin, dextran, polypeptides, etc.). Embodiments according to the present invention include embodiments wherein the radiolabeling of the SWIR-emitting particles for PET contrast are part of the composition host material (e.g., F¹⁸) present within the SWIR-emitting particles (i.e., not an additional layer) and embodiments wherein the radiolabel (e.g., O¹⁵) can be included as part of the shell composition (e.g., glucose, albumin, dextran, polypeptide, etc.). When the host material is radiolabeled, the multimodal nanoparticles of the present invention simultaneously serve three roles as a radiolabel, contrast agent and SWIR-emitting nanoparticle.

The present invention thus provides multimodal imaging methods where an optical imaging method is combined with one or more imaging methods selected from MRI, X-ray radiography, CT, PET, and the like.

While most rare earth elements can be excited to some extent by NIR light and emit to some extent in the SWIR window, rare earth-doped particle phosphors doped with ytterbium (Yb) or neodymium (Nd), and one or more elements selected from thulium (Tm), erbium (Er), praseodymium (Pr), dysprosium (Dy) and holmium (Ho) are preferred. In certain embodiments, suitable rare earth dopant schemes include Nd, Nd—Tm, Yb—Er, Yb—Tm, Yb—Pr and Yb—Ho. Furthermore, combinations of more than two rare earth dopants can be used, which include without limitation the following: Yb—Er—Tm, Yb—Pr—Tm—Er, Yb—Ho—Pr and Yb—Ho—Tm. In certain particularly preferred embodiments the host is CeF₃ and the rare earth element is selected from Pr, Nd, Yb, Er and combinations of two or more thereof. In addition, an undoped shell (>1.5 nm) surrounding the rare earth doped host may be used to reduce any emission quenching effects that typically arise from surface chemical functional groups (e.g. hydroxyl or alkyl).

The aqueous stability, particle dispersion/solubility, biocompatibility and functionality can be tailored by encapsulating rare earth nanoparticles ((RE)NPs) with suitable macromolecules (e.g. deoxyribonucleic acid, ribonucleic acid, proteins, glycoproteins), polypeptides, polysaccharides, polymers, or exosomes. Some factors that will affect the dispersion or solubility of these composites are the chemical functional group expressed on the surface, and the hydrophilicity and surface charge of the polypeptides and polymers. In addition, the size of the composites can be varied by controlling the polypeptide or polymer coating on the REs (forming differently sized (RE)ANS) to further modulate the in vivo biodistribution. Closely tied to this modification is varying the size of the RE particles themselves, in order to regulate biodistribution and clearance. For example, the albumin coating on the rare earth-containing particles enables greater and improved bioavailability and biodistribution when introduced into a mouse displaying melanoma lesions. These composites can be of different sizes (e.g. nanoscale or microscale) and morphologies (e.g. spheres, rods, platelets, prisms, cubes, acicular).

Examples of suitable macromolecules, polypeptides, polysaccharides or polymers that impart similar benefits as seen with albumin include without limitation, poly-L-lysine, poly-D-lysine, poly-ethylene glycol [PEG], poly-2-hydroxyethyl aspartamide, poly(d,l-lactide-co-glycolide) [PLGA], poly(methyl methacrylate) [PMMA], poly(N-isopropylacrylamide), poly(amidoamine) [PAMAM], poly(ethyleneimine), poly(lactic acid), polycaprolactone, dextran, alginates, chitosan, transferrin, collagenase, polydopamine, 1,2-distearoyl-sn-glycero-3-phosphoethanolamine-N-[amino(polyethylene glycol)-2000 [DSPE-PEG], and gelatin. The macromolecules, polypeptides, polysaccharides or polymers are attached to rare earth-containing particles through either physical or chemical bonds (e.g., covalent, van der Waals, ionic, electrostatic, hydrogen bonds). Encapsulation techniques can include coacervation, coprecipitation, solvent evaporation, interfacial polymerization, emulsion, reverse microemulsion, electroporation, thermal shock, saponin-assisted loading, diffusion-based methods (e.g., dialysis, sonication), transfection, and hot melt processes. The method of executing the formulation is crucial to the final composite properties and function.

These composites can be further modified through the conjugation of compounds such as antibodies and peptides, which can target tumor receptors, as well as serve as a carrier of various therapeutic agents. Upon administration in vivo, the composites are distributed throughout the organism, with the targeting ligands directing distribution to the desired target site. A plurality of targeting ligands that target a plurality of receptors can be employed, thereby increasing the specificity of delivery to both the site and the specific cells or tissues at the site. Examples of targeting ligands include, without limitation: (1) Herceptin, which preferentially binds to the HER2/neu and folate receptors; (2) Glutamic acid-Proline-Proline-Threonine (EPPT) peptide, which preferentially binds to underblycosylated MUC-1 tumor antigen (uMUC-1), a common feature of numerous epithelial cell adenocarcinomas of breast, pancreas, colon/rectum, lungs, prostate, and stomach. The adaptability of the ligand or antibody conjugation procedure means that the type, number and combinations of targeting moieties on the surface of (RE)ANS can be modified easily, further improving their tumor localization and influencing their biodistribution. Besides detecting primary tumor sites, these composites can be tailored by modifying the surface chemistry to enable identification of secondary or metastatic tumor locations and/or circulating cells and lesions. Finally, the excitation and/or emission wavelengths of the REs can be used for the purposes of eliciting a therapeutic response, such as with releasing a therapeutic agent or triggering the activation of a therapeutic agent within the (RE)ANS.

### Applications

### Contrast Agent for Non-Invasive Medical Imaging Using Non-Ionizing, Low Energy Sources

Cross-sectional 2-dimensional (2D) or 3-dimensional (3D) images of an object around a single axis of rotation can be generated using current medical imaging techniques like X-ray computed tomography (CT) and magnetic resonance imaging (MRI). Harmful side effects associated with the use of the high-energy radiation sources have limited its utility in medical diagnostics. Besides using high-energy radiation sources, intravascular contrast agents are required for both imaging modalities to improve the image quality (signal and resolution). The contrast agents are necessary to differentiate between adjacent soft tissues and organs, or to distinguish diseased tissue, such as a tumor mass, from the surrounding normal tissues. However, most current contrast agents have several limitations, including short imaging times due to rapid renal clearance, renal toxicity, allergic reactions, and vascular permeation.

In one embodiment of the invention, using a low photon energy, non-ionizing infrared light source and exploiting the NIR and SWIR biological windows, infrared imaging potentially offers a significantly safer and less invasive route to providing images of the body, its organs, and other internal structures for medical diagnostic purposes. However, for infrared imaging technology to be implemented, contrast agents to distinguish the various tissues, organs and tumor masses are needed. Having the infrared emitting composites introduced here will enable the successful implementation of infrared imaging. Furthermore, the infrared-emitting composites can also enable the non-invasive monitoring of the treatment of tumors, and provide details on the tumor architecture (e.g., vascular density).

In a further embodiment of the invention, color contrast or “multi-color” imaging can be enabled by utilizing composites that emit at different wavelengths. Color contrast is achieved by encoding the composites with different types of rare earth-containing particles that will provide each composite with a different emission wavelength. In addition, each differently-emitting composite is tailored with varying surface chemistries to direct the composite to a specific site of interest and thus enable assignment of different colors to each site. Subsequently, the “multi-color” imaging system will enable rapid identification of various sites of interests (e.g., dead vs. healthy tissues, diseased vs. healthy tissues, benign vs. malignant) simultaneously.

Image-Guides Interventions (Surgical and Non-Surgical)

In one embodiment of the invention, imaging techniques can be used to guide the insertion of small instruments and tools through the body to identify and treat a medical disorder without requiring conventional surgery. The image-guided procedures may be conducted for either diagnostic (e.g., angiogram) or treatment (e.g., angioplasty) purposes. The infrared-emitting composites can be injected to serve as vascular tracing agents to better guide surgeons performing these procedures. Images can be collected over the course of the procedure itself to provide real time information on the vasculature, and consequently significantly reduce the risk of potential undesirable complications.

Dual Functionality as Imaging Contrast Agent/Probe and a Drug Carrier

In another embodiment of the invention, the infrared-emitting composites can also serve as a drug carrier. Drug release can be either controlled by degradation rate (i.e. crosslinking density) of polymer/polypeptide or triggered by visible wavelength emissions from rare earth nanoparticles. Together with the use of the infrared imaging technology, the drug carrier penetration and treatment efficacy can be monitored. This can lead to rapid, longitudinal evaluation of drug carrier properties which can be used to optimize carrier design.

Drug delivery carriers such as coated nanoparticles strive to improve the bioavailability and, ultimately, the therapeutic action of a drug through a number of means. Drug carriers aid in increasing the aqueous solubility of poorly soluble drugs, reducing drug clearance by the reticuloendothelial system and offering a surface which can be modified with disease-targeting moieties minimizing systematic drug distribution, and ultimately leading to enhanced drug concentration at a diseased site. However, engineering an effective drug delivery vector requires an understanding of how a carrier's behavior and biodistribution in vivo are influenced by their design features. Enabling drug carriers to be visualized and subsequently tracked in real-time in vivo would provide researchers with a more thorough understand of these parameters and lead the way towards rationally engineered drug delivery.

Coatings, such as albumin nanoshells, have been shown to be capable of encapsulating and releasing small molecule drugs (<800 Da, organic compounds), nucleic acid polymers (such as antisense oligonucleotides, short interfering RNA, aptamers, etc.), plasmids, proteins and peptides, antibodies and polymers to numerous cell lines and in vivo models.

In a further embodiment of the invention, in addition to visualization, the visible wavelength emission of the rare earth nanoparticles can be used as a stimulus to induce structural or chemical changes within the drug delivery carrier or drug itself, enabling release and activation of the therapeutic agent. Designing drug carrier with energy- or light-responsive material coupled to rare earth nanoparticles enable release and therapeutic action to be triggered and controlled in an on-off manner at diseased sites.

Dual Functionality as Imaging Contrast Agent/Probe and Gene Carrier

In another embodiment of the invention related to the drug carrier functionality, the infrared-emitting composites can be further employed as a gene carrier. In a potential disease treatment method, genes are inserted, altered, or removed to correct defective genes that are responsible for disease development Undesired side effects and safety concerns associated with viral vectors, such as acute immune response, immunogenicity, and insertion mutagenesis, have limited the application of gene therapy. Using non-viral vectors provides a relatively safe approach to increase or decrease the expression of a specific gene using DNA or antisense sequences. For gene delivery using composites, the extended long chain deoxyribonucleic acid (DNA) or ribonucleic acid (RNA) molecules are condensed to reduce the occupied spatial volume. Surface modification techniques are used to introduce chemical functional groups to enable particles to tightly bind with plasmid DNA and serve as a gene delivery carrier. Using the composites as a gene delivery carrier also prevents DNA from being degraded by environmental enzymes.

### Dual Functionality as Imaging Contrast Agent/Probe and Agent for Photodynamic Therapy

Typically, photodynamic therapy (PDT) is a minimally invasive treatment that destroys target cells in the presence of oxygen when visible light irradiates a photosensitizer (e.g., porphyrins), generating highly reactive singlet oxygen that then attacks the cellular target. The use of photosensitizers excited by visible light has thus far limited the use of PDT to tissues accessible with a light source. Current clinical applications include the treatment of solid tumors of the skin, lungs, esophagus, bladder, head, neck, and the like. In another embodiment of the invention, the visible wavelength emissions from rare earth-containing particles can be used for the purposes of eliciting a therapeutic response, such as with releasing a therapeutic agent or triggering the activation of a therapeutic agent within the (RE)ANS.

In a modification to conventional photodynamic therapy method, the phototoxic properties of high photon energy ultraviolet light are harnessed to enable similar therapeutic functions without the use of photosensitizers. The optical properties of rare earth-containing particles are tuned to favor ultraviolet emissions for the implementation of this concept.

Dual Functionality as Imaging Contrast Agent/Probe and Hyperthermia Agent

Local hyperthermia (or thermal ablation) is used to heat a very small area like a tumor. It creates very high temperatures that can kill cells, coagulate proteins, and destroy blood vessels. Radiofrequency ablation (RFA) which uses high-energy radio waves, is the most commonly used type of local hyperthermia. A thin, needle-like probe is introduced into the tumor for a short time (˜10 to 15 min), where probe placement is guided using ultrasound or CT scans. The probe generates a high-frequency current that creates heat (˜50-100° C.) and destroys the cells within a certain area.

In a further embodiment of the invention, the infrared-emitting composites can be used to deliver a therapeutic dose of heat by using moderately low exposures of externally applied near-infrared light. During hyperthermia therapy, the power of the near-infrared light incident upon a selected, desired area can be increased above the operating powers used to enable imaging. The efficacy of hyperthermia treatment can be modulated using these infrared-emitting composites by tuning the emissions from rare earth-containing particles to favor mid-infrared to far-infrared emissions that will generate heat when absorbed by tissues.

Tracking of Implantable Scaffolds and Devices and Sensors

In another embodiment of the invention, these composites can be used as a coating on medical implants, scaffolds or devices, such as neurovascular or endovascular stents, to allow the functionality of these implants to be monitored. By integrating the composites into biomedical devices, guided implantation of the devices can be performed (analogous to X-ray guided implantation of emerging radiopaque stents), and the progressive changes in the integrity and remodeling of implants can be monitored and evaluated in patients non-invasively.

Tissue Targeting for Imaging of Pathologic Lesions and Plaques

In yet another embodiment of the invention, these composites can be developed with appropriate coating for preferential targeting to areas of vascular inflammation or fibrosis, and thus be used to guide the development of diagnostic strategies for imaging of atherosclerotic plaques, fibrotic and diseased tissues and lesions and vascular aneurysms, to cite just a few examples, and to track the onset of emerging chronic conditions such as neurodegenerative disorders and metabolic diseases.

In Vivo and Ex Vivo Diagnosis and Disease Screening

In still another embodiment of the invention, these infrared-emitting composites can be further modified through the conjugation of compounds such as antibodies and peptides, which can target tumor receptors or diseased lesions. Subsequently, the modified composites will be localized at the targeted sites. The differential accumulation of particles will enable target-specific in vivo molecular imaging for early screening and diagnosis of diseases. Furthermore, certain circulating cells and pathogens (e.g., metastatic cancer cells, viruses, bacteria) can also be identified using the same concept.

The afore-mentioned embodiment can be further extended to the application of infrared emitting composites for ex vivo diagnosis and screening. Body fluids (e.g., blood, saliva, urine) are analyzed for certain circulating cells and pathogens (e.g., metastatic cancer cells, viruses, bacteria) using various biochemical and histochemical assay platforms. Examples of the biochemical assay testing platforms can include but are not limited to “lab-on-a-chip” and microarrays.

In addition, histopathological examination can be performed on tissue biopsies utilizing the infrared emission of the composites. Following composite administration into a patient or in vivo model, a sample of a tissue of interest can be removed, sectioned and viewed under using a conventional microscope to identify the presence and location of the nanoparticles. For example, the nanoparticle emission can be used as a tracer to identify where the particles are located in organs such as the liver, spleen and kidneys, as well as how widespread and homogenous they are in a tumor. Since vasculature can be visualized with the particles, irregular patterns in blood flow, commonly exhibited with tumors, can potentially be identified with this system.

One embodiment of the present invention is directed to a method of non-invasive infrared imaging, including the steps of:
(a) administering a composition containing infrared-emitting nanoparticles including rare earth elements, wherein the nanoparticles are encapsulated with a biocompatible matrix to form downconverting coated nanoparticles tuned to optimize short-wavelength infrared (SWIR) emission; and
(b) irradiating with infrared radiation, wherein both excitation and emission spectra of the coated nanoparticles are in the infrared region.

Another embodiment of the invention is directed to a method of image-guided biomedical intervention, including the steps of:
(a) administering a composition containing infrared-emitting nanoparticles including rare earth elements, wherein the nanoparticles are encapsulated with a biocompatible matrix to form downconverting coated nanoparticles tuned to optimize short-wavelength infrared (SWIR) emission; and
(b) irradiating with infrared radiation, wherein both excitation and emission spectra of the coated nanoparticles are in the infrared region.

Still another embodiment of the invention is directed to a method of drug tracking and delivery, including the steps of:
(a) administering a drug composition containing infrared-emitting nanoparticles including rare earth elements, wherein the nanoparticles are encapsulated with a biocompatible matrix to form downconverting coated nanoparticles tuned to optimize short-wavelength infrared (SWIR) emission; and
(b) irradiating with infrared radiation, wherein both excitation and emission spectra of the coated nanoparticles are in the infrared region.

Another embodiment of the invention is directed to a method of improving the biocompatibility and/or reducing the toxicity and/or side effects of a drug and/or imaging agent, including the step of encapsulating the drug and/or imaging agent with a biocompatible matrix.

Yet another embodiment of the invention is directed to a method of biologically targeting a drug and/or imaging agent, including the step of encapsulating the drug and/or imaging agent with a biocompatible matrix.

For any of the above methods, a preferred biocompatible matrix includes human serum albumin (HSA). In addition, for any of the above methods, the biocompatible matrix can further include a pharmaceutical agent, and/or a targeting molecule which directs the coated nanoparticle to a biological target. A preferred targeting molecule is cyclic arginine-glycine-aspartic acid (cRGD) tripeptide.

For the methods including nanoparticles, the nanoparticles preferably include CeF₃ doped with one or more rare earth elements selected from Yb, Nd, Pm, Sm, Eu, Gd, Tb, Tm, Er, Pr, Dy and Ho and Ho. Most preferably the rare earth elements are selected from Pr, Nd, Yb and Er. In addition, for the methods including nanoparticles, the nanoparticles can include one or more MRI, CT and X-ray radiography contrast elements. The contrast element can be an additional component of the particle; or the host material or RE dopant can be selected from materials and REs that function as contrast agents. In one embodiment the contrast agent is selected from Er, Dy, La, Ce, Pm, Sm, Eu, Gd, Tb, and Lu. These elements can be part of the SWIR-emitting composition or part of the shell composition (e.g., glucose, albumin, dextran, polypeptides, etc.) Er and Dy can serve a dual role in both optical and MRI/CT/X-ray imaging. The contrast element provides a composition suitable for use as a multimodal imaging agent. Preferably the nanoparticles are about 20 to about 500 nm in size, preferably about 50 nm or greater, or about 100 nm or greater, or about 200 nm or greater, or about 300 nm or greater, or about 400 nm or greater, or about 500 nm. Alternatively, for some applications the particles can be microscale size, for example about 100 μm or about 10 μm or about 1 μm.

For MRI-based methods, in addition to rare earth elements such as Gd and Dy, Mn can also serve as a dopant or constituent of the SWIR/MRI multimodal nanoparticles. For CT-based methods, Ba is also a useful contrast element.

Compositions according to the present invention can also be radiolabeled for PET imaging. The radiolabeling of the SWIR-emitting particles for PET contrast can be part of the composition (e.g., F¹⁸) present within the SWIR-emitting particles (i.e., not an additional layer). The radiolabel (e.g., O¹⁵) can also be included as part of the shell composition (e.g., glucose, albumin, dextran, polypeptides, etc.).

The multimodal imaging scheme can thus be selected from optical/MRI, optical/CT, optical/X-ray imaging, optical/PET and combinations thereof. For example, one multi-modal combination according to the present invention combines optical, MRI, and PET imaging. Another embodiment combines optical, CT and PET imaging.

Yet another embodiment of the invention is directed to a composition for biomedical applications, including infrared-emitting nanoparticles including rare earth-elements, wherein the nanoparticles are encapsulated with a biocompatible matrix to form downconverting coated nanoparticles tuned to optimize short-wavelength infrared (SWIR) emission. Preferably the biocompatible matrix is human serum albumin (HSA). Also, the biocompatible matrix can further include a pharmaceutical agent, and/or a targeting molecule which directs the coated nanoparticle to a biological target. A preferred targeting molecule includes cyclic arginine-glycine-aspartic acid (cRGD) tripeptide.

Multimodal imaging compositions according to the present invention can be formulated with pharmaceutically acceptable carriers suitable for and conventionally used for imaging agent delivery.

As above, the nanoparticles are a low phonon energy halide host doped with one or more rare earth elements selected from Yb, Nd, Pm, Sm, Eu, Gd, Tb, Tm, Er, Pr, Dy and Ho. Compositions according to the present invention include, but are not limited to compositions based on halide hosts selected from BaF₂, NaYF₄, YF₃, LaF₃, CeF₃, CaF₂ and CsCdBr₃. In one embodiment, the nanoparticles include CeF₃ host doped with one or more rare earth elements selected from Yb, Nd, Pm, Sm, Eu, Gd, Tb, Tm, Er, Pr, Dy and Ho. Most preferably the rare earth elements are selected from Pr, Nd, Yb and Er. In addition, the nanoparticles can further include one or more contrast agents and the contrast agents can be selected from one or more elements selected from Er, Dy, La, Ce, Pm, Sm, Eu, Gd, Tb, and Lu, including embodiments where Er and/or Dy are the sole optical and contrast elements used. The contrast elements can be part of the SWIR-emitting composition or part of the shell composition (e.g., glucose, albumin, dextran, polypeptides, etc.)

Compositions according to the present invention can also be radio-labeled for PRT imaging. The radiolabeling of the SWIR-emitting particles for PET contrast can be part of the composition (e.g., F¹⁸) present within the SWIR-emitting particles (i.e., not an additional layer). The radiolabel (e.g., O¹⁵) can also be included as part of the shell composition (e.g., glucose, albumin, dextran, polypeptides, etc.)

### EXAMPLES

### Example 1. Small Animal SWIR-Imaging System Using NIR Excitation

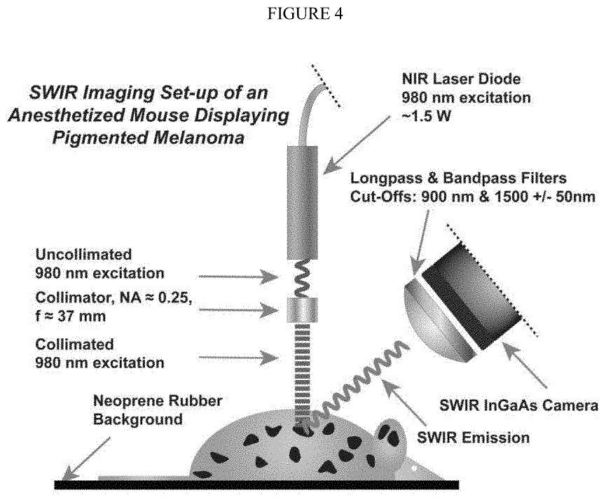

A prototype of a small animal SWIR-imaging system was designed and built to demonstrate of the utility of our composites for non-invasive SWIR imaging. The in-house SWIR-imaging prototype consists of a fiber-coupled NIR laser photodiode which operates at 980 nm and 1.4-1.5 W, and a thermoelectric-cooled Indium Gallium Arsenide (InGaAs) SWIR camera with a imaging range from 800 to 1700 nm (see FIG. 4). FIG. 4 shows a SWIR imaging set-up of an anesthetized mouse displaying pigmented melanoma following (RE)ANS injection. Excitation of the mouse was performed using a 980 nm laser diode equipped with a collimator operating at approximately 1.4-1.5 W. Emission was captured using a SWIR InGaAs camera equipped with two sets of filters (longpass-900 nm and bandpass-1500 nm+/−50 nm) to ensure the elimination of the excitation light from the captured images. Black Neo-prene rubber was used as the background for the mouse to rest on in order to provide an infrared black background in order to enhance the signal-to-noise ratio for the collected emission.

A collimator is attached to the excitation fiber to enable a uniform and constant excitation beam radius which is independent of the distance between source and animal subject. During the imaging, the excitation fiber is held at an arbitrary distance above the animal and slowly scanned across the animal's body. Any SWIR emissions are then simultaneously captured in real time by the SWIR camera that is positioned in a fixed height above the animal. These videos are captured at ˜19-22 frames per second (i.e. imaging exposure time ˜45-53 msec). Optical filters were fitted onto the SWIR camera to eliminate imaging of the NIR excitation source to confidently determine that only the SWIR emissions were captured. An incandescent Xe flashlight was used to provide backlight to partially resolve the location of the mouse. Finally, black Neoprene rubber or a latex-paint-coated cardboard was used as the background surface on which the mouse is placed to reduce the amount of ambient reflected light and produce a favorable signal-to-noise ratio. Compared to another similarly functioning system which uses a 20 W pulsed NIR source and liquid nitrogen cooled SWIR camera with exposure times in the hundreds of milliseconds for the imaging of SWIR-emitting carbon nanotubes, the above prototype operates at significantly lower excitation power with better detector sensitivity.

### Example 2. Detection of NIR-to-SWIR Fluorescence In Vivo

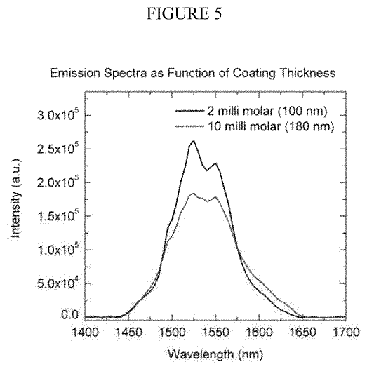

Variously sized (RE)ANS were introduced through intraperitoneal and intravenous administration into rodent models. The nanoparticles were imaged using an SWIR camera to capture the SWIR emission of the particles following their fluorescent excitation at 980 nm. The SWIR emission around 1500 nm of the REs following NIR excitation was confirmed for the ANS-encapsulated composites of the RE's (FIG. 5). This is consistent with the previous reports that, in addition to the traditional “optical imaging window” of NIR between 650 to 950 nm, there lies another second “window” in the SWIR region of the spectrum between 1000 to 1700 nm mimicking the improved signal-to-noise ratios for seen in traditional NIR imaging.

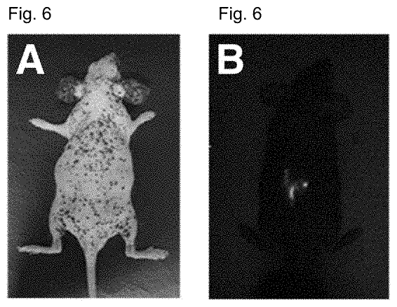

In order to examine the biodistribution of the (RE)ANS, a melanoma model was used. These mice develop melanoma spontaneously around the ears, snout and anus followed by lesions on their skin and metastases in their lymph nodes. Many of these tumors become highly vascularized over time. Two strains of the mice were utilized: ones with highly pigmented melanoma lesions and ones without this distinguishing pigment. Using the SWIR emission of 100 nm (RE)ANS, the presence of the nanoparticles in tissues including liver, vasculature and tumors, was successfully identified in the mice expressing pigmented lesions following either intravenous (IV) or intraperitoneal (IP) injection of the nanoparticles (FIGS. 6A and 6B). During the SWIR imaging, no visible wavelength emissions were observed at the sites where SWIR emissions were observed. Any visible wavelength emissions were most likely attenuated by the skin, tissues or blood.

FIG. 5 displays the SWIR emission spectra of variously sized (RE)ANS following 980 nm excitation. Both the 2 millimolar (100 nm) and 10 millimolar (180 nm) (RE)ANSs display emission in the SWIR, indicating the RE emissive properties are retained after albumin encapsulation. FIGS. 6A and 6B shows a picture of a mouse exhibiting melanoma lesions injected with 100 μl of (RE)ANS (2.0 mg RE ml⁻¹) via the peritoneum and imaged for SWIR emission after 30 minutes. A digital camera image of the mouse prior to injection show the black, pigmented lesions are located throughout the mouse body particularly on the ears and back (A). Numerous sites throughout the animal showed SWIR emission when the 980 nm excitation laser diode irradiated a particular region. Sites on the animal's back showed what appears to be emission of the nanoparticles in structures resembling vasculature (B), indicating the ability to rapidly screen and track nanoparticles bio-distribution in real time.

Thus, the proof-of-concept of the superior in vivo biophotonic properties of the nanocomposites of the invention has been established. This also demonstrates that albumin encapsulation enables greater biodistribution of the RE compared with the uncoated RE.

### Example 3. Superiority of Biodistribution of RE-Composites Over Naked RE's

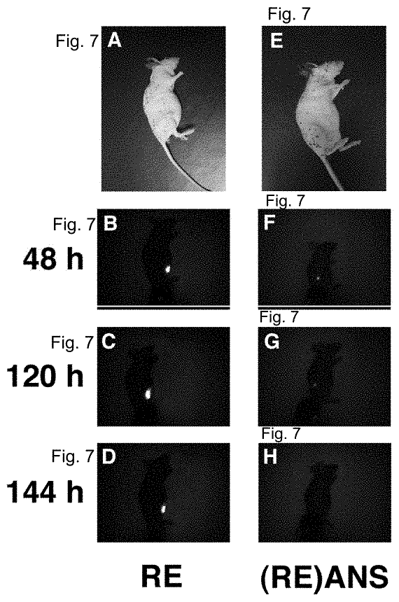

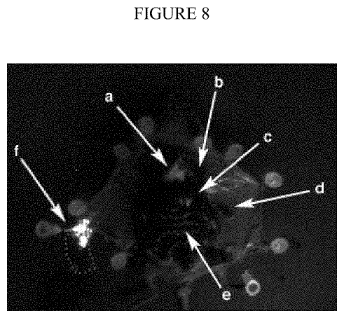

Striking differences in biodistribution following IP injection of REs or (RE)ANS (FIGS. 7A-7H) were observed. While the (RE)ANS in the peritoneal cavity progressively diminish from the site of injection over time, accumulating and clearing from different organs and tumors (FIGS. 7F-7H), uncoated RE particles do not distribute from the site of injection even after 7 days (FIGS. 7B-7D. After dissecting the animal most of the uncoated REs were found to be located in the IP cavity sac, a highly vascularized structure lining the abdomen and known to be the first point of entry into the blood stream of compounds introduced via IP injection (FIG. 8). This confirms that albumin encapsulation facilitates the absorption of the coated RE particles into the bloodstream, enabling improved biodistribution compared to the REs alone.

FIG. 7 shows pictures of mice exhibiting melanoma lesions injected with 100 μl of REs and (RE)ANS (2.0 mg RE ml⁻¹) via the peritoneum. Digital camera images of the mice prior to injection show the black, pigmented lesions are located throughout the mice bodies particularly on their ears, anus and back (FIGS. 7A and 7E). After 48 h following IP injection, both mice injected with REs (FIG. 7B) and (RE)ANS (FIG. 7F) exhibit SWIR emission around the site of injection when the 980 nm excitation laser diode irradiates the region. Over time, however, the emission of the (RE)ANS diminishes (FIGS. 7G and 7H), indicated the nanoparticles are clearing the injection site, in contrast to the REs (FIGS. 7C and 7D), which appear to remain at the site of injection. FIG. 8 shows a picture of a mouse exhibiting melanoma dissected after 168 h following IP injection of REs (100 μl at 2.0 mg RE ml⁻¹). Although most organs such as the lungs (a), heart (b), liver (c), lymph node(s) (d) and intestines (e) do not exhibit SWIR fluorescence, the intraperitoneal cavity sac (f and structure dissected from the animal outlined), a highly vascularized structure lining the abdomen and known to be the first point of entry into the blood stream of compounds introduced via IP injection. This indicates that the REs are unable to enter into circulation following their injection into the peritoneum.

### Example 4. Tissue Targeting and Multifunctional Drug Carrier Platforms

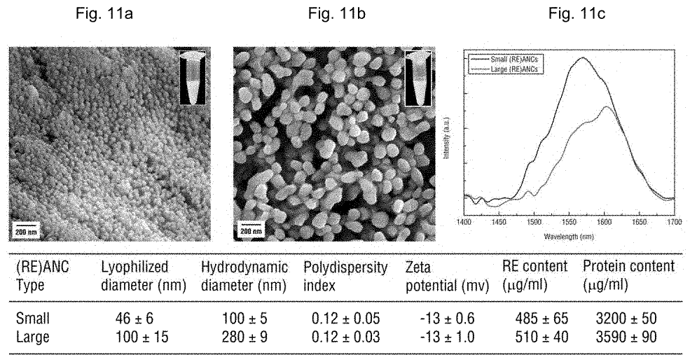

Adjusting the particle size of the REs and the presentation of specific receptor targeting moieties on the (RE)ANS can lead to improved and directed tissue-targeting. FIGS. 11A-11C show physical (FIGS. 11A and 11B) and optical (FIG. 11C) properties of (RE)ANCs. SEM images of both small (a) and large (b) (RE)ANCs show uniform sub-100 nm spherical particles. Both sizes of (RE)ANCs retain the SWIR emission of the encapsulated REs, exhibiting peak emission between 1550-1600 nm following 980 nm excitation (c). Based on microscopy imaging, small and large lyophilized (RE)ANCs exhibit an average size of 46 nm and 100 nm which appears to swell when hydrated to 100 and 280 nm, respectively. DLS measurements confirm a low polydispersity and heterogeneity of size. Furthermore, both formulations show negative zeta potentials in PBS (pH 7.4), with the HSA content increasing for larger (RE)ANCs ±S.D. Various pharmacologic factors and/or targeting molecules may be incorporated into the (RE)ANS thus creating a drug delivery vector that can be tracked in vivo using NIR-to-SWIR optical imaging.

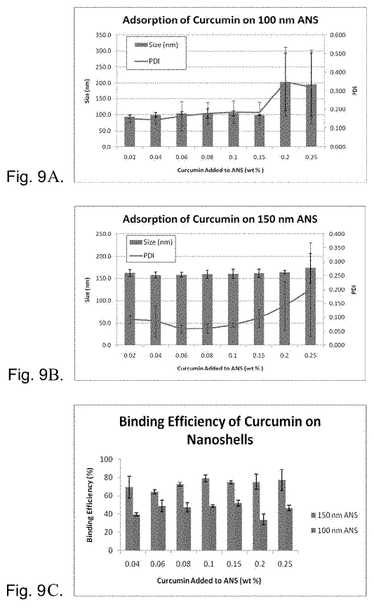

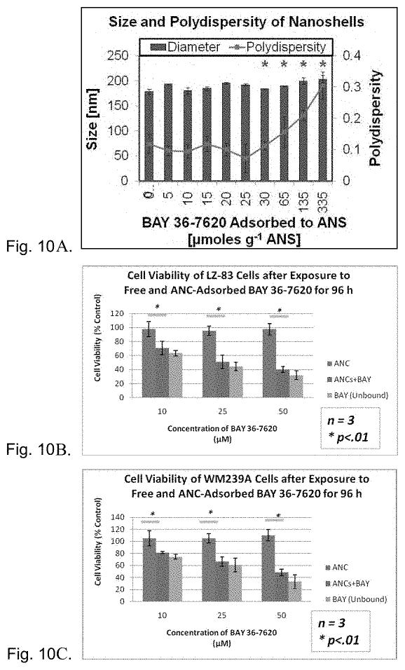

One embodiment of the invention is directed to incorporating various cancer therapeutic agents into the (RE)ANS coating. Monomeric albumin is known to contain two primary drug binding sites as well as numerous secondary binding sites. It is thus possible to utilize these intrinsic sites to confine compounds known to be effective against cancers such as melanoma onto the (RE)ANS. The maximum drug loading quantities for compounds such as curcumin (FIGS. 9A and 9B), a naturally derived compound shown to have therapeutic properties in a variety of cancers, and BAY 36-7620 (FIG. 9C), a receptor antagonist shown to be effective against melanoma, have been determined. Furthermore, it has been demonstrated that the in vitro efficacy of these drugs is retained following association with the nanoparticles (FIGS. 10A-10C). Thus, the optical properties of the (RE)ANS can be used to track the delivery of drug in vivo, enabling longitudinal evaluation of drug biodistribution and delivery optimization.

FIGS. 9A-9C shows drug loading of variously sized ANS with curcumin (FIGS. 9A and 9B) and binding efficiency of loading (FIG. 9C). Both 100 and 150 nm ANS begin to display progress increase in polydispersity when loaded with >0.15 wt % of curcumin, indicating maximum loading. This corresponds to greater than 50% binding efficiency for the 100 nm ANS and approximately 75% efficiency for the 150 nm ANS. FIGS. 10A-10C shows drug loading of ANS with BAY 36-7620 (FIG. 10A) and cytotoxicity assay of LZ-83 murine (FIG. 10B) and WM239A human (FIG. 10C) melanoma cells exposed to ANSs with and without BAY 36-7620, a glutamate receptor antagonist shown to induce cell death in numerous melanoma cell lines. Through sizing and polydispersity measurements performed using DLS, 25 μmoles BAY 36-7620 g⁻¹ nanoshells can be adsorbed onto the surface of the nanoshells. When exposed to melanoma cell lines, although ANSs alone do not induce notable cytotoxicity in the melanoma cell lines, the ANSs loaded with BAY 36-7620 show comparable cytotoxicity to BAY 36-7620 alone after 96 h, indicating that the drug's activity is retained even after incorporation into the nanoparticles.

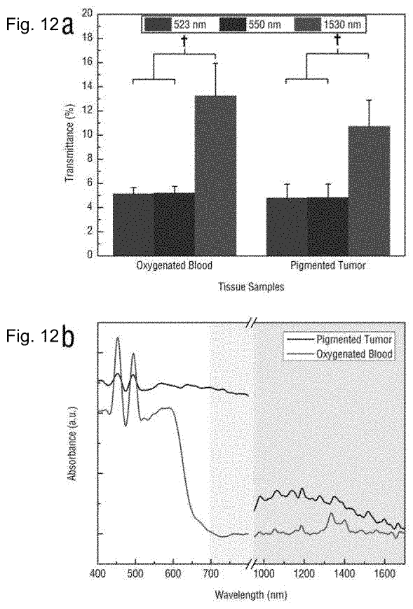

FIGS. 12A and 12B show the transmission efficiency and absorbance of SWIR and visible RE emissions through blood and pigmented tumor samples. RE(Er) nanoparticles with emission in both the visible and SWIR transmit through blood and tumor samples approximately 3- and 2-fold greater, respectively, in the SWIR (1530 nm) than compared to their peak emissions in the visible (523 and 550 nm) (FIG. 12A). Furthermore, the absorbance of the melanin present in the tumors exhibits a minimum beyond 900 nm, while blood shows an absorbance minimum beyond 700 nm (FIG. 12B). Note the break in the spectra from 800 to 900 nm is due to the spectroscope detector change. The shaded region represents the NIR while blue the SWIR. ±SE; †, P<0.01 Student's t-test.

Furthermore, the natural ability of albumin to bind to multiple classes of compounds makes possible simultaneous, multi-drug delivery. Numerous therapeutic compounds have been shown to work in a synergistic fashion when combined with other therapeutic agents, eliciting greater responses than if the drugs were acting alone. Delivering two or more drugs simultaneously via the nanocarriers, in which the drugs act on multiple or individual targets, may provide a greater disease therapy.

The above examples, taken together with the following claims are intended to be representative of the invention, and not intended to limit it in any way.

We claim:

1. A composition for biomedical applications, comprising a plurality of infrared-emitting particles comprising rare earth-elements that emit in the short-wavelength infrared (SWIR) spectrum, said particles optionally further comprising a radiolabel for PET imaging, or one or more contrast elements for MRI, CT or X-ray radiographic imaging, or both a radiolabel and a contrast element,
wherein said infrared-emitting particles are directly encapsulated with a shell comprising one or more encapsulants selected from the group consisting of polypeptides, polysaccharides, biocompatible polymers, and exosomes, to form spherical down-converting microcapsules comprising a plurality of said infrared-emitting particles, wherein said infrared-emitting particles have a size between 2 nm and 10 micrometers, wherein said microcapsules have a capsule size between 10 nm and 100 micrometers, and wherein said infrared-emitting particles have a relative size permitting the plurality of infrared-emitting particles to be loaded into said microcapsules.

2. The composition of claim 1, wherein said one or more of the polypeptides, polysaccharides and biocompatible polymers of said shell is selected from the group consisting of poly-L-lysine, poly-D-lysine, polyethylene glycol, poly-2-hydroxyethyl aspartamide, poly(D,L-lactide-co-glycolide), poly(methyl methacrylate), poly(N-isopropylacrylamide), poly(amidoamine), poly(ethyleneimine), polylactic acid, polycaprolactone, dextran, alginates, chitosan, transferrin, collagenase, polydopamine, 1,2-distearoyl-sn-glycero-3-phosphoethanolamine-N-[amino(polyethylene glycol)-2000 (DSPE-PEG), and gelatin.

3. The composition of claim 1, wherein said shell comprises the biocompatible polymer poly(ethyleneimine).

4. The composition of claim 1, wherein said shell further comprises a pharmaceutical agent.

5. The composition of claim 1, wherein said shell further comprises one or more targeting molecules that direct said encapsulated infrared-emitting particles to a biological target.

6. The composition of claim 1, wherein said infrared-emitting particles comprise a low phonon energy halide host.

7. The composition of claim 6, wherein said low phonon energy halide host is selected from the group consisting of CeF₃, BaF₂, NaYF₄, YF₃, LaF₃, CaF₂, CsCdBr₃, SrFCl and SrF₂.

8. The composition of claim 1, wherein said infra-red emitting particles comprise a low or high phonon energy host doped with one or more rare earth elements selected from the group consisting of Yb, Nd, Pm, Sm, Eu, Gd, Tb, Tm, Er, Pr, Dy and Ho, wherein said host is transparent to the emission wavelength or wavelengths of the rare earth element dopant.

9. The composition of claim 1, wherein said one or more optional contrast elements are selected from the group consisting of Er, Dy, La, Ce, Pm, Sm, Eu, Gd, Tb, and Lu.

10. The composition of claim 1, wherein said infrared-emitting particles are a factor of about 10 or more smaller than said microcapsules.

11. The composition of claim 1, wherein the infrared-emitting particle loading in said microcapsules ranges from 0.004 wt % to 94 wt %.

12. A method of non-invasive multimodal imaging of a subject, comprising the steps of:
a) administering to a subject a composition according to claim 1, and
b) subjecting the subject to a multimodal imaging scheme selected from the group consisting of optical imaging in combination with an imaging method selected from the group consisting of MRI, CT, X-ray radiography and combinations thereof, wherein said composition comprises a contrast agent, and/or a PET imaging method, wherein said composition comprises a radiolabel.

13. The method of claim 12, wherein the shell of said composition comprises human serum albumin (HSA).

14. The method of claim 12, wherein the shell of said composition further comprises one or more targeting molecules that direct said encapsulated infrared-emitting particles to a biological target.

15. The method of claim 14, wherein the targeting molecule comprises cyclic arginine-glycine-aspartic acid (cRGD) tripeptide.

16. The method of claim 32, wherein the SWIR-emitting particles of said composition comprise a low phonon energy halide host.

17. The method of claim 12, wherein said low phonon energy halide host is selected from the group consisting of CeF₃, BaF₂, NaYF₄, YF₃, LaF₃, CaF₂, CsCdBr₃, SrFCl and SrF₂.

18. The method of claim 17, wherein said low phonon energy halide host is CeF₃ doped with said one or more SWIR-emitting rare earth elements selected from the group consisting of Yb, Nd, Pm, Sm, Eu, Gd, Tb, Tm, Er, Pr, Dy and Ho.

19. The method of claim 12, wherein the multimodal imaging scheme is optical/MRI, optical/CT and/or optical/X-ray radiographic imaging, and said particles further comprise a contrast element selected from the group consisting of Er, Dy, La, Ce, Pm, Sm, Eu, Gd, Tb, and Lu.

20. The method of claim 12, wherein the multimodal imaging scheme is optical/PET.
